(19) United States
(12) Patent Application Publication
(10) Pub. No.: US 2017/0119504 A1
(43) Pub. Date: May 4, 2017

(54) ORTHODONTIC PLATE FIXED TO MANDIBULAR RAMUS

(71) Applicant: THE CATHOLIC UNIVERSITY OF KOREA INDUSTRY-ACADEMIC COOPERATION FOUNDATION, Seoul (KR)

(72) Inventor: Yoon Ah KOOK, Seoul (KR)

(21) Appl. No.: 15/235,888

(22) Filed: Aug. 12, 2016

(30) Foreign Application Priority Data
Oct. 30, 2015 (KR) 10-2015-0152025

(51) Int. Cl. A61C 8/00; A61B 17/86; A61C 7/22; A61B 17/80

(52) U.S. Cl. CPC A61C 8/0096; A61B 17/8071; A61B 17/86; A61C 7/22

(57) ABSTRACT

Provided is an orthodontic plate to be fixed to a mandibular ramus, the orthodontic plate including a fixing part implanted into a retromolar fossa portion of a mandible in a mouth, the retromolar fossa portion being near a molar; a hook part connected to the fixing part, and dynamically connected to orthodontic appliances placed at lower teeth for mandibular orthodontics; and a connection part connecting the fixing part and the hook part, the connection part projecting and being sloped from a front end surface including the fixing part toward a front end surface including the hook part, so as to allow the hook part to be positioned without being interrupted by the lower teeth.

### CROSS-REFERENCE TO RELATED APPLICATION

This application claims the benefit of Korean Patent Application No. 10-2015-0152025, filed on Oct. 30, 2015, in the Korean Intellectual Property Office, the disclosure of which is incorporated herein in its entirety by reference.

### BACKGROUND

1. Field

The present disclosure relates to an orthodontic plate fixed to a mandibular ramus, and more particularly, to an orthodontic plate fixed to the mandibular ramus and having an improved structure to be implanted into a mandible without cutting soft tissue, thereby increasing convenience in mandibular orthodontics.

2. Description of the Related Art

Teeth positioned in a mouth are divided into lower teeth and upper teeth, and in this regard, the upper teeth are held by the maxilla and the lower teeth are held by a mandible.

The mandible referred to as a lower jawbone is connected to the skull via a mandibular joint. In early childhood, the mandible is divided into left and right bones and then the left and right bones are combined at a center portion into a single bone. The front portion of the mandible is referred to as a mandibular body, and the rear portion of the mandible is referred to as the mandibular ramus.

The mandible and the maxilla of the human body are bones that most involve mastication, and misalignment thereof causes a disorder of masticatory muscles which results in chronic pain, facial asymmetry, and pain in the neck and the upper part of the shoulder, therefore, orthodontic treatments are necessary.

In general, ramus fixing for mandibular orthodontics is performed in a manner that soft tissue around a retromolar fossa portion is cut and a plate is implanted therein, and then a portion of the plate which is exposed from the soft tissue is connected to orthodontic appliances placed at the lower teeth.

However, the aforementioned treatment appliances and methods cause inconvenience of cutting the soft tissue and connecting the orthodontic appliances with the plate.

### SUMMARY

In order to solve the aforementioned problems, the disclosure provides an orthodontic plate to be fixed to a mandibular ramus, thereby increasing convenience in mandibular orthodontics by improving a connection structure with respect to orthodontic appliances placed at lower teeth.

The disclosure also provides an orthodontic plate to be fixed to a mandibular ramus by being implanted into a retromolar fossa portion without cutting soft tissue.

Additional aspects will be set forth in part in the description which follows and, in part, will be apparent from the description, or may be learned by practice of the presented embodiments.

According to an aspect of an embodiment, an orthodontic plate to be fixed to a mandibular ramus includes a fixing part implanted into a retromolar fossa portion of a mandible in a mouth, the retromolar fossa portion being near a molar; a hook part connected to the fixing part, and dynamically connected to orthodontic appliances placed at lower teeth for mandibular orthodontics; and a connection part connecting the fixing part and the hook part, the connection part projecting and being sloped from a front end surface including the fixing part toward a front end surface including the hook part, so as to allow the hook part to be positioned without being interrupted by the lower teeth.

In order to reduce a contact area between the fixing part and soft tissue of the retromolar fossa portion, the fixing part may have a tapered outer surface having an external diameter that gradually increases as the outer surface is farther from the contact area.

The hook part may be connected to the connection part to be rotatable relative to the connection part.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a perspective view of an orthodontic plate to be fixed to a mandibular ramus, according to an embodiment;

FIG. 2 illustrates a perspective view of the orthodontic plate in a usage state, according to an embodiment;

FIG. 3 illustrates a front view and partial cross-sectional view of the orthodontic plate, according to an embodiment;

FIG. 4 illustrates a perspective view of an orthodontic plate to be fixed to a mandibular ramus, in a usage state, according to another embodiment; and

FIG. 5 illustrates a front view and partial cross-sectional view of an orthodontic plate, according to another embodiment.

### DETAILED DESCRIPTION

An orthodontic plate to be fixed to a mandibular ramus will now be made in detail to embodiments, examples of which are illustrated in the accompanying drawings.

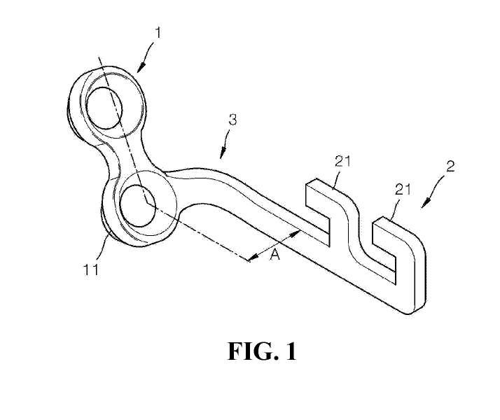

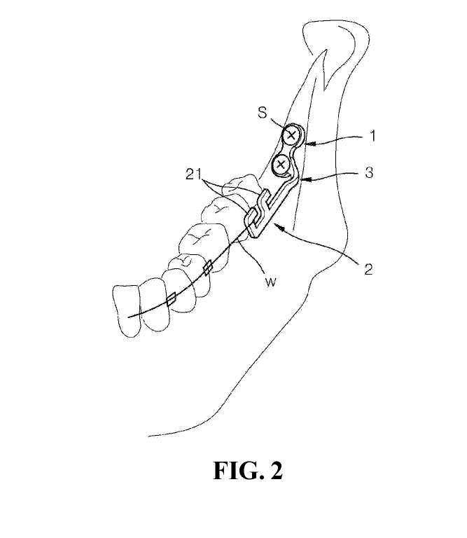

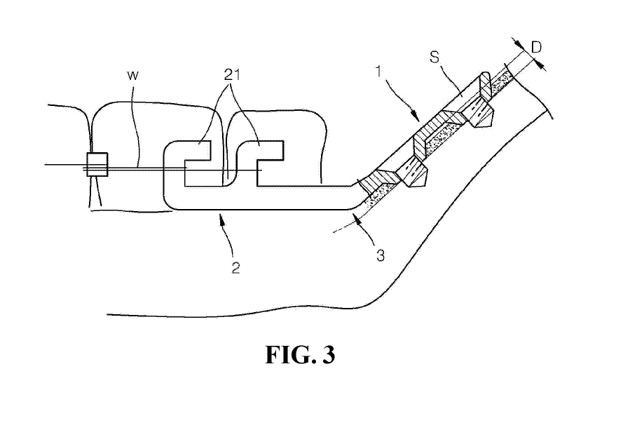

FIG. 1 illustrates a perspective view of an orthodontic plate to be fixed to a mandibular ramus, according to an embodiment, FIG. 2 illustrates a perspective view of the orthodontic plate in a usage state, according to an embodiment, and FIG. 3 illustrates a front view and partial cross-sectional view of the orthodontic plate, according to an embodiment.

As illustrated in the drawings, the orthodontic plate to be fixed to the mandibular ramus includes a fixing part 1, a hook part 2, and a connection part 3.

The fixing part 1 is implanted into a retromolar fossa portion of a mandible in a mouth. Although the fixing part 1 may be fixedly implanted under the skin by cutting soft tissue of the retromolar fossa portion, it is preferable that the fixing part 1 is fixed to a bone located under the soft tissue by using a screw S without cutting the soft tissue, due to inconvenience of cutting the soft tissue.

In the present embodiment, the screw S passes through each of a pair of holes formed in the fixing part 1 and is coupled to one side of the mandible, so that the fixing part 1 may be firmly supported on the mandible.

The hook part 2 having one or more hooks is connected to the fixing part 1, and is dynamically connected to orthodontic appliances placed at the lower teeth for mandibular orthodontics. That is, a tension-adjustable wire W is connected with the hooks of the hook part 2 so as to apply tension to the orthodontic appliances.

In this regard, since the connection between the hook part 2 and the orthodontic appliances, which is requested for the mandibular orthodontics, is achieved by hooking the tension-adjustable wire W on the hooks of the hook part 2, the connection may be easily and conveniently performed.

The connection part 3 is placed between the fixing part 1 and the hook part 2 so as to connect the fixing part 1 and the hook part 2 to each other.

According to the disclosure, the orthodontic plate to be fixed to the mandibular ramus for mandibular orthodontics has a structure for connection with the orthodontic appliances that are placed at the lower teeth, at a plate for mandibular orthodontic treatments, so that dental appliances for the mandibular orthodontics may be further conveniently placed.

As described above, the fixing part 1 may be fixedly implanted under the skin by cutting the soft tissue. However, due to inconvenience in cutting the soft tissue, the fixing part 1 may be fixedly implanted without cutting the soft tissue.

To do so, instead of cutting the soft tissue, the screw S passes through the fixing part 1 and then is coupled to the bone through the soft tissue, so that the orthodontic plate to be fixed to the mandibular ramus may be fixed.

Therefore, it is required to inhibit inflammation of the soft tissue which occurs due to penetration of the screw S, and in order to inhibit the inflammation, it is required to make a thickness of the screw S be as small as possible and to make a contact area between the fixing part 1 and the soft tissue be small.

In the present embodiment, in order to significantly reduce a contact area between the fixing part 1 and soft tissue corresponding to a groove in a rear side of a molar, as illustrated in FIG. 3, the fixing part 1 has a tapered outer surface 11 having an external diameter that gradually increases as the tapered outer surface 11 is farther from the contact. A length D of the outer surface 11 which corresponds to a length of the screw S may be between about 4 mm and about 6 mm.

For example, if the length D of the outer surface 11 is less than 4 mm, the screen S having a standard size may not be properly screwed to the bone, and if the length D of the outer surface 11 is greater than 6 mm, bearing power of the screw S with respect to the fixing part 1 may deteriorate.

As illustrated in FIG. 1, the connection part 3 projects (refer to A of FIG. 1) and is sloped from a front end surface including the fixing part 1 to a front end surface including the hook part 2, so that the hook part 2 may be positioned without being interrupted by the lower teeth.

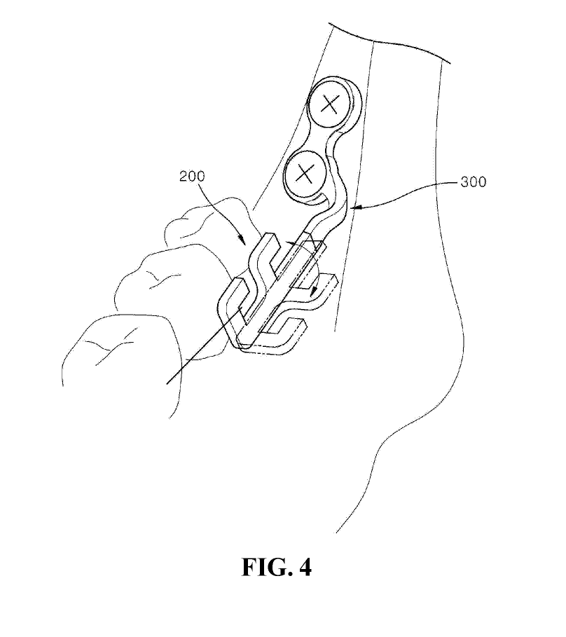

Hereinafter, an orthodontic plate to be fixed to a mandibular ramus according to another embodiment will now be described in detail with reference to FIG. 4.

FIG. 4 illustrates a perspective view of an orthodontic plate to be fixed to a mandibular ramus, the orthodontic plate being in a usage state, according to another embodiment.

A hook part 200 applied to the present embodiment is connected to a connection part 300 so as to be rotational relative to the connection part 300.

The present embodiment allows the hook part 200 to rotate with respect to the connection part 300 by taking into account an intraoral structure that differs according to persons or a tension-adjustable level required according to persons.

That is, by adjusting an angle of the hook part 200 that is hooked to a tension-adjustable wire of orthodontic appliances, a structure having two hooks may replace a structure having three or four hooks.

According to the present embodiment, by using a simple structure in which the hook part 200 is rotational relative to the connection part 300, the present embodiment performs the same function as other products having a larger number of hooks than the number of hooks of the present embodiment, so that it is possible to actively apply various tension values of different patients to the present embodiment.

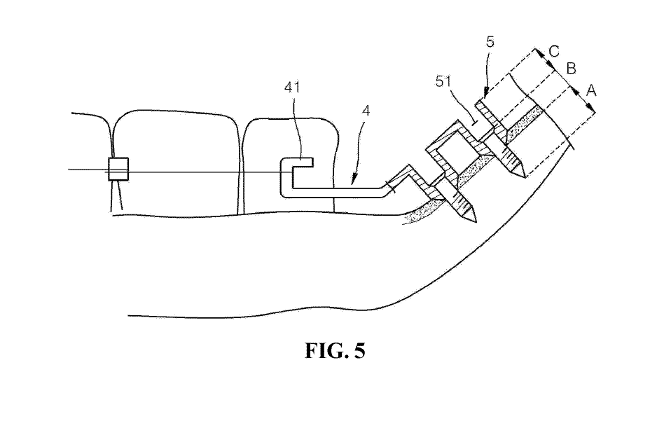

FIG. 5 illustrates a front view and partial cross-sectional view of an orthodontic plate, according to another embodiment.

In the present embodiment of FIG. 5, one hook 41 is formed at an end of a hook part 4, an empty space 51 is arranged in a fixing part 5, and a head of a screw is positioned at a bottom of the empty space 51 so that a distance between a top of the head and a top of the fixing part 5 is relatively large.

In the present embodiment, a length A of the screw which is implanted into a bone is about 5 mm, a length B of the screw which is implanted from the top of the head of the screw into soft tissue is about 3 mm, and a length C between the top of the head of the screw and the top of the fixing part 5 is about 4 mm.

In the present embodiment having the aforementioned structure, a length of the screw which is implanted into the bone and soft tissue of a human body is increased to reinforce fixation power of the screw with respect to the bone, and an overall center of mass moves from a top portion of the fixing part 5 toward a bottom portion of the fixing part 5 due to the empty space 51 in the fixing part 5, so that structural stability may be achieved.

The orthodontic plate for mandibular orthodontics according to the one or more embodiments has the structure for connection with the orthodontic appliances that are placed at the lower teeth, so that dental-procedure instruments for the mandibular orthodontics may be further conveniently placed.

While one or more exemplary embodiments have been described with reference to the figures, it will be understood by those of ordinary skill in the art that various changes in form and details may be made therein without departing from the spirit and scope as defined by the following claims.

What is claimed is:

1. An orthodontic plate to be fixed to a mandibular ramus, the orthodontic plate comprising:
a fixing part implanted into a retromolar fossa portion of a mandible in a mouth, the retromolar fossa portion being near a molar;
a hook part connected to the fixing part, and dynamically connected to orthodontic appliances placed at lower teeth for mandibular orthodontics; and
a connection part connecting the fixing part and the hook part, the connection part projecting and being sloped from a front end surface comprising the fixing part toward a front end surface comprising the hook part, so as to allow the hook part to be positioned without being interrupted by the lower teeth.

2. The orthodontic plate of claim 1, wherein, in order to reduce a contact area between the fixing part and soft tissue of the retromolar fossa portion, the fixing part has a tapered outer surface having an external diameter that gradually increases as the outer surface is farther from the contact area.

3. The orthodontic plate of claim 1, wherein the hook part is connected to the connection part to be rotatable relative to the connection part.

4. The orthodontic plate of claim 1, wherein the fixing part accommodates a screw for fixing the fixing part and has an empty space arranged between an upper surface of the fixing part and a surface of a head of the screw.
